(19) United States
(12) Patent Application Publication
(10) Pub. No.: US 2019/0184140 A1
(43) Pub. Date: Jun. 20, 2019

(54) APPARATUS FOR USE WITH CATHETERS

(71) Applicant: David DeIulio, Lafayette, LA (US)

(72) Inventor: David DeIulio, Lafayette, LA (US)

(21) Appl. No.: 16/326,220

(22) PCT Filed: Jul. 21, 2017
(86) PCT No.: PCT/US2017/043274

§ 371 Date: Feb. 17, 2019

Related U.S. Application Data
(60) Provisional application No. 62/376,699, filed on Aug. 18, 2016.

(51) Int. Cl. A61M 25/02

(52) U.S. Cl. CPC A61M 25/02; A61M 2025/028; A61M 2025/0286

(57) ABSTRACT

An apparatus comprising a first component; a second component; and a locking element adapted to selectively couple the first and second components together, wherein the locking element is rotatable between a first position and a second position, wherein the apparatus is adapted to secure a catheter between the first and second components when the locking element is in the first position, and wherein the catheter is removable from the apparatus when the locking element is in the second position.

### FIELD OF THE DISCLOSURE

The present disclosure relates to catheters, and more particularly to an apparatus for use with catheters.

### RELATED ART

Catheters are typically affixed to the skin of a body by adhesive tape, the use of which may be undesirable, in particular for neonates having underdeveloped skin which may become damaged upon application or removal of adhesive tape. The medical industry continues to demand improved methods and apparatuses for securing catheters.

### BRIEF DESCRIPTION OF THE DRAWINGS

Embodiments are illustrated by way of example and are not limited in the accompanying figures.

FIG. 1 includes a top perspective view of an apparatus in accordance with an embodiment, as seen in a closed configuration.

FIG. 2 includes a top perspective view of the apparatus in accordance with an embodiment, as seen in an open configuration.

FIG. 3 includes a top view of the apparatus in accordance with an embodiment.

FIG. 4 includes a top perspective view of an apparatus in accordance with an embodiment.

FIG. 5 includes a bottom perspective view of the first component of the apparatus in accordance with an embodiment.

FIG. 6 includes a top perspective view of a second component of the apparatus in accordance with an embodiment.

FIG. 7 includes a perspective view of a locking element of the apparatus in accordance with an embodiment.

FIG. 8 includes a bottom perspective view of the second component of the apparatus in accordance with an embodiment.

FIG. 9 includes a cross-sectional view of the second component as seen along Line A-A of FIG. 8 in accordance with an embodiment.

FIG. 10 includes a perspective view of a locking element of the apparatus in accordance with an embodiment.

FIG. 11 includes a cross-sectional view of a portion of the apparatus in accordance with an embodiment.

FIG. 12 includes a bottom perspective view of a first component of the apparatus in accordance with another embodiment.

Skilled artisans appreciate that elements in the figures are illustrated for simplicity and clarity and have not necessarily been drawn to scale. For example, the dimensions of some of the elements in the figures may be exaggerated relative to other elements to help to improve understanding of embodiments of the invention. Embodiments illustrated in separate figures may be provided in combination in a single embodiment, and conversely, various features that are, for brevity, illustrated in the context of a single embodiment, may also be provided separately or in any subcombination.

### DETAILED DESCRIPTION

The following description in combination with the figures is provided to assist in understanding the teachings disclosed herein. The following description will focus on specific implementations and embodiments of the teachings. This focus is provided to assist in describing the teachings and should not be interpreted as a limitation on the scope or applicability of the teachings.

As used herein, the terms “comprises,” “comprising,” “includes,” “including,” “has,” “having,” or any other variation thereof, are intended to cover a non-exclusion inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

The use of “a” or “an” is employed to describe elements and components described herein. This is done merely for convenience and to give a general sense of the scope of the invention. This description should be read to include one or at least one and the singular also includes the plurality, or vice versa, unless it is clear that it is meant otherwise.

Unless otherwise defined, all technical and scientific terms used herein have the same meaning as commonly understood by one of ordinary skill in the art to which this invention belongs. The materials, methods, and examples are illustrative only and not intended to be limiting. To the extent not described herein, many details regarding specific materials and processing acts are conventional and may be found in textbooks and other sources within the medical device arts.

An apparatus in accordance with one or more of the embodiments described herein can generally include a first component and a second component. The first and second components can be selectively coupled together by a locking element moveable between a first position and a second position. In an embodiment, the first and second positions can be angularly (e.g., rotationally) offset from one another. In another embodiment, the first and second positions can be longitudinally offset from one another. In a further embodiment, the first and second positions can be angularly and longitudinally offset from one another.

The apparatus is adapted to receive a catheter, such as an umbilical catheter. In an embodiment, the apparatus is adapted to receive a plurality of catheters. For example, the apparatus can receive at least two catheters, or at least three catheters, or at least four catheters, or at least five catheters. In an embodiment, the apparatus can selectively couple the catheter(s) within a receiving portion of the apparatus and secure the catheter at a fixed, or relatively fixed, position with respect to a body of the apparatus. In an embodiment, the receiving portion includes a recess or channel. In a particular embodiment, part of the receiving portion can be disposed on the first component and part of the receiving portion can be disposed on the second component. In a more particular embodiment, half, or approximately half, of the receiving portion can be disposed on the first component and half, or approximately half, of the receiving portion can be disposed on the second component.

The catheter can be secured to the apparatus via compressive force applied by the receiving portions of the first and second components. In a particular embodiment, the catheter is disposed between the first and second components. In a more particular embodiment, the catheter is compressed between the receiving portions of the first and second components. Typically, compression between the two components (e.g., along the Y-axis) can create deformation of a catheter along a perpendicular axis (e.g., along the X-axis). The receiving portion can be adapted to prevent deformation of the catheter in the perpendicular axis. That is, the receiving portion can be adapted to mitigate perpendicular axis deformation. When in the second (closed) positioned, the apparatus can permit desirable compressive force along the circumference of the catheter, effectively forming a uniform 360°, or substantially 360°, contact-loading point along the catheter wall. That is, force applied by the apparatus to the catheter can vary by no greater than 50%, or no greater than 25%, or no greater than 10%, or no greater than 5%, or no greater than 1% at any two points measured around the circumference of the catheter. In a particular instance, 360°, or substantially 360°, permits maximum grip between the apparatus and the catheter.

In an embodiment, the first and second components are spaced apart from one another when in the second (closed) position by a gap. The gap can have a distance of at least 0.1 mm, or at least 0.2 mm, or at least 0.5 mm, or at least 1 mm, or at least 5 mm. In an embodiment, the gap is less than 50 mm, or less than 25 mm, or less than 10 mm. In a particular instance, the gap has a distance, as measured by a shortest distance between the first and second components, less than an outer circumference of the catheter, or less than 99% of the outer circumference of the catheter, or less than 75% of the outer circumference of the catheter, or less than 50% of the outer circumference of the catheter, or less than 25% of the outer circumference of the catheter, or less than 10% of the outer circumference of the catheter.

In an embodiment, the apparatus is adapted to provide substantially equal compressive force to the catheter, as measured around the circumference of the catheter. That is, the force provided by the apparatus to the catheter can be generally uniform as measured at different locations around the circumference of the catheter. In an embodiment, the force provided by the apparatus to the catheter can be within +/−5 pounds per square inch (PSI), or within +/−4 PSI, or within +/−3 PSI, or within +/−2 PSI, or within +/−1 PSI at 90% or more of the circumferential locations around the catheter. The force of the apparatus on the catheter can thus be generally uniform as measured around the circumference of the catheter. Uniform circumferential force along the outer surface of the catheter can prevent deformation of the catheter lumen during secured engagement with the apparatus 100, thereby preventing occlusions and restrictions of the lumen during use.

The apparatus can be secured to the body (e.g., of a neonate) to prevent relative movement between the apparatus and the body. An apparatus in accordance with one or more of the embodiments described herein generally permits selective attachment of a catheter at a generally fixed position relative to a body. In an embodiment, the apparatus mitigates undesirable movement of the catheter relative to the body. This may be particularly suitable for use with umbilical catheters used to administer fluids and drugs and conduct sampling during a neonate's duration in a hospital. Such umbilical catheters must remain precisely positioned for days (or weeks) to prevent injuring the patient.

Referring to FIGS. 1 to 4, an apparatus 100 in accordance with an embodiment generally includes a first component 102 and a second component 104. The first and second components 102 and 104 can be selectively coupled together by a locking element 106.

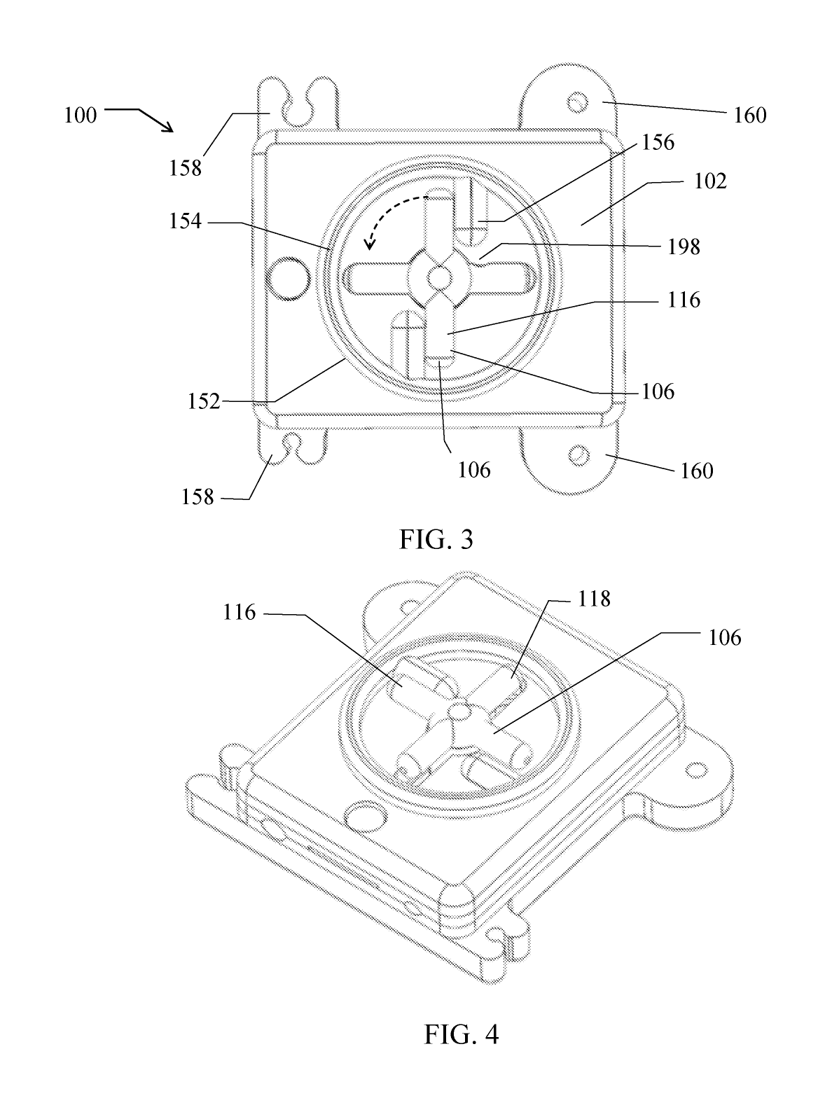

In an embodiment, the locking element 106 extends at least partially through an opening 108 in the first component 102. In another embodiment, the locking element 106 extends at least partially through an opening 110 in the second component 104. In a further embodiment, the locking element 106 extends at least partially through the openings 108 and 110 of the first and second components 102 and 104, respectively. As illustrated, the openings 108 and 110 can extend through the entire thickness of the first and second components 102 and 104, respectively. In another embodiment, at least one of the openings 108 or 110 can extend less than the entire thickness of the first or second component 102 or 104. The locking element 106 may not extend through the entire thickness of the apparatus 100. In a particular embodiment, the locking element 106 can be secured within at least one of the openings 108 and 110 such that the locking element 106 is not removable, or readily removable, therefrom. As used with respect to the locking element 106, “readily removable” refers to a condition whereby the locking element 106 will remain within the opening 108 or 110 when not biased. The locking element 106 can be secured within the at least one opening 108 or 110, for example, by a tab or projection 198 (see also FIG. 3) that extends into the opening and is shaped to prevent translation of the locking element 106 through the opening 108 or 110.

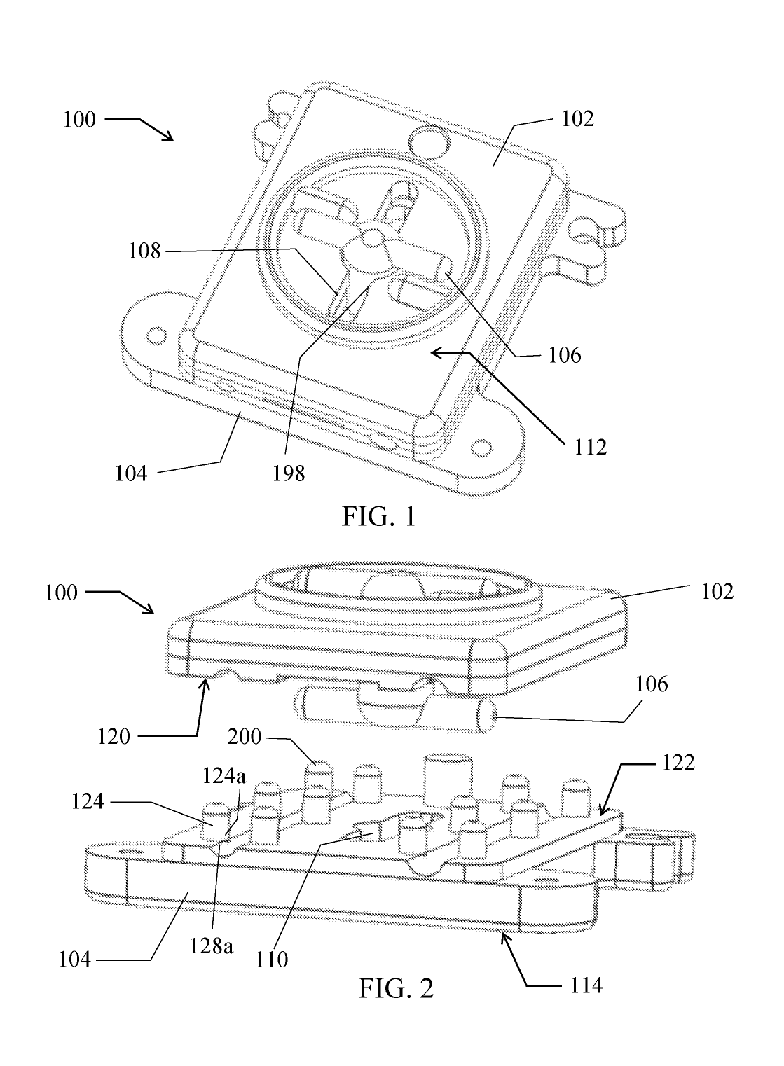

As illustrated in FIG. 1, the locking element 106 can contact an outer surface 112 of the first component 102. The locking element 106 can also contact an outer surface 114 (FIG. 2) of the second component 104. The locking element 106 can provide a compressive force between the first and second components 102 and 104 (e.g., between surfaces 112 and 114) to lock the first component 102 and the second component 104 together. In a particular embodiment, the locking element 106 can contact both outer surfaces 112 and 114 and provide a compressive force therebetween. In a non-illustrated embodiment, the locking element 106 can contact only one of the outer surfaces 112 or 114 and secure with the other component 102 or 104 at a location spaced apart from the respective outer surface 112 or 114. In yet another non-illustrated embodiment, the locking element 106 can be spaced apart from both outer surfaces 112 and 114 while securing the first and second components 102 and 104 together.

FIGS. 3 and 4 illustrate the locking element 106 in both a first position 116 and a second position 118. The locking element 106 can be adjustable between the first position 116 and the second position 118. The apparatus 100 can be selectively locked when the locking element 106 is in the first position 116. As used herein, “selectively locked” refers to a condition whereby the first and second components 102 and 104 are coupled relative to one another such that a catheter (not illustrated) can be coupled at a fixed location relative to the apparatus 100. In the locked position, the apparatus 100 can generally prevent relative movement between the catheter and the first and second components 102 and 104. In an embodiment, the apparatus 100 can be selectively locked without a catheter positioned within the apparatus 100. In another embodiment, the apparatus 100 can only be selectively locked when a catheter is positioned within the apparatus 100. That is, the apparatus 100 may not lock, or may not effectively lock, without a catheter positioned within the apparatus 100. This can occur, for example, in embodiments adapted to include a gap between the first and second components in the locked position.

In an embodiment, the first position 116 is angularly offset from the second position 118 by at least 1°, or at least 2°, or at least 3°, or at least 4°, or at least 5°, or at least 10°, or at least 20°, or at least 30°, or at least 45°, or at least 90°. In another embodiment, the first position 116 is angularly offset from the second position 118 by less than 360°, or no greater than 270°, or no greater than 180°, or no greater than 135°. In an embodiment, the first and second positions 116 and 118 are angularly offset from one another by an angle in a range of 1° and 359°, or in a range of 5° and 315°, or in a range of 45° and 270°, or in a range of 60° and 180°. As illustrated in FIGS. 3 and 4, the first and second positions 116 and 118 can be angularly offset by approximately 90°, however such angle is not required as the apparatus 100 may be locked at offset angles other than (e.g., less than) 90°. In the illustrated embodiment, locking can occur, for example, at 10°, or at 15°, or at 20°, or at 30°, or at 45°, or at any other rotation other than 90° as measured from the first position 116.

In another embodiment, the first position 116 is offset from the second position 118 by a translational distance (e.g., a longitudinal distance perpendicular to the outer surface 112 or 114) of at least 0.1 mm, or at least 0.2 mm, or at least 0.5 mm, or at least 1 mm, or at least 2 mm, or at least 3 mm, or at least 4 mm, or at least 5 mm, or at least 10 mm, or at least 20 mm. In another embodiment, the first position 116 is offset form the second position 118 by a distance of no greater than 100 mm, or no greater than 50 mm, or no greater than 20 mm. In an embodiment, the first and second positions 116 and 118 are offset form one another by a distance in a range of 0.1 mm and 100 mm, or in a range of 0.2 mm and 10 mm, or in a range of 0.3 mm and 5 mm. In such a manner, the locking element 106 can be moved between locked and unlocked configurations by translating the locking element 106 in at least one of an X-, Y-, or Z-direction.

In an embodiment, the first position 116 is translationally and angularly offset from the second position 118 by a distance and an angular displacement. That is, the locking element 106 is both rotated and translated when the locking element 106 is moved between locked and unlocked configurations. In a particular instance, the user can generate rotational force to rotationally translate the locking element 106. Translational movement can occur as the result of the design of the apparatus (e.g., inclusion of a ramp which causes translational movement in response to rotational movement).

Referring to FIG. 2, the first component 102 can include an inner surface 120 and the second component 104 can include an inner surface 122. In a particular embodiment, at least one, such as both, of the inner surfaces 120 and 122 can be generally planar. As used herein, “generally planar” refers to a surface that deviates from a best fit plane by no greater than 50 mm at any location, or no greater than 25 mm at any location, or no greater than 10 mm at any location, or no greater than 5 mm at any location, or no greater than 4 mm at any location, or no greater than 3 mm at any location, or no greater than 2 mm at any location, or no greater than 1 mm at any location. In an embodiment, at least one, such as both, of the inner surfaces 120 and 122 can be planar such that no portion of the inner surfaces 120 and 122 deviate from a best fit plane by more than 0.5 mm, or more than 0.3 mm, or more than 0.1 mm. In an embodiment, at least one of the inner surfaces 120 and 122 can include one or more of ridges, concavities, such as dimples, recesses, secondary channels, or other indentions.

In an embodiment, the first and second components 102 and 104 can be brought together to secure a catheter therebetween by translating the first and second components 102 and 104 together in a direction generally perpendicular to the inner surfaces 120 and 122. As used herein, “generally perpendicular” refers to an angle in a range of 85° and 95°, or in a range of 86° and 94°, or in a range of 87° and 93°, or in a range of 88° and 92°, or in a range of 89° and 91°. In another embodiment, the first and second components 102 and 104 are engaged by translating the first and second components 102 and 104 together in a direction perpendicular (in a range of 89.5° and 90.5°) to the inner surfaces 120 and 122.

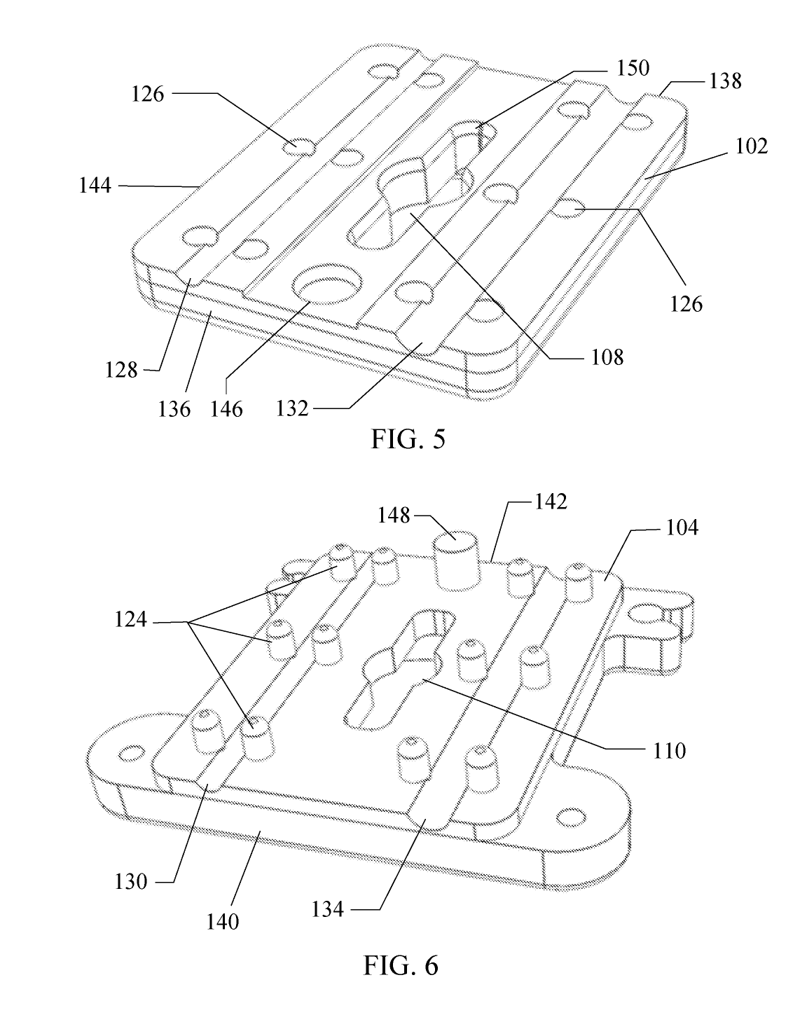

In an embodiment, one or more posts 124 can extend from at least one of the inner surfaces 120 or 122. One or more recesses 126 (FIG. 5) can extend into the other of the inner surfaces 120 or 122 to receive a complementary post 124. In an embodiment, all of the one or more posts 124 extend from a same inner surface 120 or 122. For example, as illustrated in FIG. 2, all of the posts 124 can extend from the inner surface 122 of the second component 104. In another embodiment, at least one of the posts 124 can extend from the inner surface 120 of the first component 102 and at least one of the posts 124 can extend from the inner surface 122 of the second component 104. The recesses 126 can be complementary positioned along one or both of the first and second components 102 or 104 to receive the posts 124.

In an embodiment, at least two of the posts 124 can extend a same distance, as measured from the inner surface 120 or 122 (e.g., at least two of the posts 124 can have a same height). In another embodiment, at least two of the posts 124 can extend different distances, as measured with respect to one another from the inner surface 120 or 122. By way of a non-limiting example, in an embodiment at least one of the posts 124 can extend at least 0.1 mm from the inner surface 120 or 122, or at least 0.2 mm from the inner surface 120 or 122, or at least 0.5 mm from the inner surface 120 or 122, or at least 1 mm from the inner surface 120 or 122, or at least 5 mm from the inner surface 120 or 122. In another embodiment at least one of the posts 124 can extend no greater than 500 mm from the inner surface 120 or 122, or no greater than 200 mm from the inner surface 120 or 122, or no greater than 100 mm from the inner surface 120 or 122, or no greater than 50 mm from the inner surface 120 or 122.

In an embodiment, at least one of the posts 124 extends from the inner surface 120 or 122 a distance that is no less than 5% of a width of an adjacent receiving portion (described in greater detail below). The receiving portion is adapted to close fit with a catheter having a particular diameter. Thus, the at least one post 124 extends from the inner surface 120 or 122 a distance no less than 5% of the catheter to be received by the adjacent receiving portion. In a further embodiment, the at least one post 124 extends from the inner surface 120 or 122 a distance no less than 10% of the diameter of the catheter, or no less than 20% of the diameter of the catheter, or no less than 30% of the diameter of the catheter, or no less than 40% of the diameter of the catheter. In a more particular embodiment, the at least one post 124 extends from the inner surface 120 or 122 a distance no less than 50% of the diameter of the catheter, or no less than 55% of the diameter of the catheter, or no less than 60% of the diameter of the catheter, or no less than 65% of the diameter of the catheter, or no less than 70% of the diameter of the catheter, or no less than 75% of the diameter of the catheter, or no less than 80% of the diameter of the catheter, or no less than 85% of the diameter of the catheter, or no less than 90% of the diameter of the catheter, or no less than 95% of the diameter of the catheter, or no less than 99% of the diameter of the catheter. By way of a non-limiting example, the posts 124 can be adapted to receive and temporarily maintain a catheter in close proximity to an adjacent receiving portion while the apparatus 100 is not locked (e.g., when a user is inserting a second catheter into the apparatus or while the apparatus is being locked). Posts extending a distance greater than a threshold height (e.g., 50% the diameter of the catheter) may provide enhanced temporary retention capability as compared to posts extending distances less than the threshold height.

In an embodiment, at least one of the recesses 126 can extend a depth, as measured from the inner surface 120 or 122, of at least 0.1 mm, or at least 0.2 mm, or at least 0.5 mm, or at least 1 mm, or at least 5 mm. In an embodiment, at least one of the recesses 126 extends through an entire thickness of the first or second component 102 or 104. In an embodiment, the depth of at least one of the recesses 126 can be equal to the height of the complementary post 124 to be received in the at least one recess 126. In another embodiment, the depth of at least one of the recesses 126 can be different than the height of the complementary post 124 to be received in the at least one recess 126. For example, in a particular embodiment, the depth of the recess(es) 126 can be greater than the height of the post(s) 124. In another particular embodiment, the depth of the recesses 126 can be less than the height of the posts 124. In a particular instance, the depth of the recess(es) 126 can be less than the height of the post(s) 124 when the inner surfaces 120 and 122 are spaced apart when the apparatus 100 is locked.

As illustrated in FIG. 2, the posts 124 and recesses 126 can have generally cylindrical shapes. The apex 200 of at least one of the posts 124 can be rounded or otherwise tapered to facilitate easier insertion of each post 124 into a corresponding recess 126. Tapered or rounded apexes 200 can further permit easier installation of a catheter into an area between posts 124 on opposite sides of the receiving portion. In an embodiment, the corresponding recess 126 can have a rounded or otherwise tapered bottom to meet the apex 200 of the corresponding post 124. In an embodiment, the apex 200 of at least one of the posts 124 can be spaced apart from the bottom of the corresponding recess 126 when the apparatus 100 is locked. In another embodiment, the apex 200 of at least one of the posts 124 can contact, or nearly contact, the bottom of the corresponding recess 126 when the apparatus 100 is locked.

In an embodiment, at least one of the recesses 126 receives only one post 124. In another embodiment, all of the recesses 126 each receive only one post 124. That is, a ratio of posts 124 to recesses 126 [posts:recesses] can be 1:1. In an embodiment, at least one of the recesses 126 can receive at least two posts 124, or at least three posts 124, or at least four posts 124, or at least five posts 124. In an embodiment, the ratio of posts 124 to recesses 126 [posts:recesses] can be in a range of 50:1 and 1:50, or in a range of 25:1 and 1:25, or in a range of 10:1 and 1:10.

In an embodiment, the posts 124 can act as guides to position the catheter within a receiving portion 128 of the apparatus 100 during installation of the catheter within the apparatus 100. The posts 124 can assist an operator when guiding the catheter into the receiving portion 128. In certain embodiments, the receiving portion 128 has an arc length less than 180°. That is, the receiving portion 128 is less than semi-circular in cross-section. In such embodiments, the catheter may not remain within the receiving portion 128 during installation of the catheter (e.g., when locking the apparatus 100). The posts 124 can assist the operator to prevent the catheter from being accidently squeezed or kinked between the inner surfaces 120 and 122 by keeping the catheter aligned with the receiving portion 128 when the apparatus 100 is not yet fully closed.

In an embodiment, at least one of the posts 124 is disposed immediately adjacent to the receiving portion 128. In a more particular embodiment, an inner edge 124a of at least one of the posts 124 can be contiguous with an inner surface 128a of the receiving portion 128. In a further embodiment, a majority, such as all, of the posts 126 are disposed immediately adjacent to the receiving portion 128. In an even further embodiment, inner edges of a majority, such as all, of the posts are contiguous with the inner surface of the receiving portion. In a particular embodiment, a distance between posts 124 on opposite sides of the receiving portion 128, as measured perpendicular to a length of the receiving portion 128, can be generally equal to the width of the receiving portion 128, as measured between edges of the receiving portion 128. The distance between posts 124 on opposite sides of the receiving portion 128 can correspond, or generally correspond, to a diameter of a catheter adapted to be received in the receiving portion 128. In another embodiment, the distance between posts 124 on opposite sides of the receiving portion 128, as measured perpendicular to the length of the receiving portion 128, can be at least 1.01 the width of the receiving portion 128, or at least 1.02 the width of the receiving portion 128, or at least 1.05 the width of the receiving portion 128, or at least 1.1 the width of the receiving portion 128. In yet another embodiment, the distance between posts 124 on opposite sides of the receiving portion 128, as measured perpendicular to the length of the receiving portion 128 at a location spaced apart from the inner surface 120 or 122, can be at no greater than 0.99 the width of the receiving portion 128.

In an embodiment, the posts 124 can be canted (e.g., not normal) relative to the inner surface 120 or 122 of the first or second component 102 or 104, respectively. By way of a non-limiting example, posts 124 on opposite sides of the receiving portion 128 can taper away from one another such that a smallest distance between opposite posts 124 is at, or adjacent to, the inner surface 120 or 122.

In an embodiment, the posts 124 are relatively rigid. In a more particular embodiment, the posts 124 are rigid and non-deformable. In a further embodiment, the posts 124 can be adapted to deform during installation of the catheter into the apparatus 100.

Referring to FIG. 6, the second component 104 can include a receiving portion 130 adapted to receive a portion of the catheter (not illustrated) generally opposite a portion adapted to be received in the receiving portion 128 of the first component 102. For example, the receiving portion 128 can receive a first circumferential portion (e.g., a first circumferential half) of the catheter and the receiving portion 130 can receive a second circumferential portion (e.g., a second circumferential half) of the catheter. In an embodiment, the receiving portions 128 and 130 together receive the catheter and provide biasing force against the catheter during compression between the first and second components 102 and 104.

In an embodiment, the first component 102 can include a second receiving portion 132 and the second component 104 can include a second receiving portion 134. The second receiving portions 132 and 134 can receive and secure a second catheter to the apparatus 100. As illustrated in FIGS. 5 and 6, in an embodiment the receiving portions 128 and 130 can have a first size and the second receiving portions 132 and 134 can have a second size different from the first size. For example, the first receiving portions 128 and 130 can have a first diameter and the second receiving portions 132 and 134 can have a second diameter. In an embodiment, the second diameter is greater than the first diameter. By way of non-limiting example, the second diameter can be at least 1.01 times greater than the first diameter, or at least 1.05 times greater than the first diameter, or at least 1.1 times greater than the first diameter, or at least 1.2 times greater than the first diameter, or at least 1.3 times greater than the first diameter, or at least 1.4 times greater than the first diameter, or at least 1.5 times greater than the first diameter. In another embodiment, the second diameter is no greater than 5 times the first diameter, or no greater than 4 times the first diameter, or no greater than 3 times the first diameter, or no greater than 2 times the first diameter. In a particular instance, the receiving portions 128 and 130 are adapted to receive a first catheter having a different size than a second catheter receivable in the second receiving portions 132 and 134.

In an embodiment, the receiving portions 128 and 130 can have the same size as the second receiving portions 132 and 134. In such a manner, the receiving portions 128 and 130 and second receiving portions 132 and 134 can both receive the same-sized catheter.

In a particular embodiment the receiving portions 128 and 130 and second receiving portions 132 and 134 can be disposed between posts 124 and recesses 126. More particularly, the receiving portions 128 and 130 and second receiving portions 132 and 134 can be immediately adjacent with the posts 124 and recesses 126. In such a manner, catheters can be readily aligned by the posts 124 and guided into the receiving portion 130 and second receiving portion 134. Further, the posts 124 can maintain the catheters at a relatively fixed location relative to the receiving portion 130 and second receiving portion 134, thus mitigating the occurrence of undesirable movement of the catheter prior to locking the apparatus 100.

In an embodiment, the receiving portions 128 and 130 can have a same or similar shape as compared to one another. For example, the receiving portions 128 and 130 can both be generally arcuate, such as hemi-circular, or polygonal. In another embodiment, the receiving portions 128 and 130 can have different shapes as compared to one another. For example, one of the receiving portions 128 or 130 can have a generally arcuate shape while the other of the receiving portions 128 or 130 has a generally polygonal shape. Similarly, the second receiving portions 132 and 134 can have same, similar, or different shapes as compared to one another. In a particular embodiment, the receiving portions 128 and 130 and second receiving portions 132 and 134 can have generally arcuate shapes. In such a manner, the receiving portions 128 and 130 and second receiving portions 132 and 134 can have a shape generally similar to the shape of the outer surface of the catheter, providing maximum contact between catheter and apparatus 100, thus increasing grip therebetween.

In an embodiment, the inner surfaces 120 and 122 of the first and second components 102 and 104 are spaced apart from one another when the apparatus 100 is in the locked configuration. For example, the inner surfaces 120 and 122 can be spaced apart by at least 0.01 mm, or at least 0.1 mm, or at least 1 mm, or at least 2 mm, or even at least 5 mm. In a particular embodiment, a portion of the catheter may be visible when viewed from a side view through the gap between the inner surfaces 120 and 122. In a more particular embodiment, a portion of the catheter may be visible along an entire, or substantially entire, length of the apparatus 100 through the gap between the inner surfaces 120 and 122. Without wishing to be bound by any particular theory, it is noted that the presence of a gap between the inner surfaces 120 and 122 in the closed configuration can allow compressive force to be transmitted only, or substantially only, to the catheter and not between the inner surfaces 120 and 122. That is, compressive force provided by the locking element 106 can be transmitted substantially to the catheter, whereas contact between the surfaces 120 and 122 in the locked configuration might prevent full transfer of compressive force thereto.

In certain embodiments, the receiving portions 128 and 130 lie along generally straight lines. As used herein, “generally straight lines” deviate from a best fit line by no greater than 10 mm, or no greater than 9 mm, or no greater than 8 mm, or no greater than 7 mm, or no greater than 6 mm, or no greater than 5 mm, or no greater than 4 mm, or no greater than 3 mm, or no greater than 2 mm, or no greater than 1 mm at any location therealong. In another embodiment, the receiving portions 128 and 130 can lie along straight lines deviating from a best fit line by no greater than 0.5 mm, or no greater than 0.1 mm. In yet a further embodiment, the receiving portions 128 and 130 can lie along curved or polygonal lines or line segments.

In an embodiment, the second receiving portions 132 and 134 can lie along generally straight lines, along straight lines, or along curved or polygonal lines or line segments. In an embodiment, the second receiving portions 132 and 134 can have a similar or same shape as the receiving portions 128 and 130. In a more particular embodiment, the second receiving portions 132 and 134 can have generally reflective shape as compared to the receiving portions 128 and 130. In another embodiment, the second receiving portions 132 and 134 can have a different shape as compared to the receiving portions 128 and 130.

In an embodiment, the receiving portions 128 and 130 and second receiving portions 132 and 134 can terminate at opposite, or generally opposite, sides of the apparatus 100. For example, as illustrated in FIG. 5, the receiving portion 128 and second receiving portion 132 terminate at opposite sides 136 and 138 of the first component 102. Similarly, as illustrated in FIG. 6, the receiving portion 130 and second receiving portion 134 can terminate at opposite sides 140 and 142. In another embodiment, the receiving portions 128 and 130, the second receiving portions 132 and 134, or a combination thereof can terminate at adjacent sides of the first and second components 102 and 104. By way of a non-illustrated example, the receiving portion 128 can terminate at sides 136 and 144.

In an embodiment, the first component 102 includes a first alignment feature 146 and the second component 104 includes a second alignment feature 148. As illustrated, the first and second alignment features 146 and 148 extend from the inner surfaces 120 and 122, respectively. The first and second alignment features 146 and 148 may be alignable with respect to one another and permit alignment of the first component 102 with the second component 104 prior to fully translating the first and second components 102 and 104 together. In an embodiment, the first and second alignment features 146 and 148 can prevent incorrect assembly of the apparatus 100. For example, in a previously described embodiment the receiving portions 128 and 130 have a different size than the second receiving portions 132 and 134. In said embodiment, improper installation of the first and second components 102 and 104 (i.e., reverse installation) may prevent closure of the apparatus 100, prevent proper seating of the catheters with respect to the first and second components 102 and 104, or prevent sufficient grip of the catheters with the apparatus 100.

While the first and second alignment features 146 and 148 are illustrated as a recess and post, respectively, in another embodiment the first and second alignment features 146 and 148 can include any other alignment protocol such as a tab and groove, a nonsymmetrical arrangement of posts 124 and recesses 126, ridges, visual indicia, any other alignment mechanism known by skilled artisans, or any combination thereof. While not illustrated, the posts 124 and recesses 126 can be positioned in a non-reflective arrangement such that the posts 124 and recesses 126 do not align when the first and second components 102 and 104 are incorrectly positioned with respect to one another.

As illustrated in FIGS. 5 and 6, in an embodiment one or both of the openings 108 and 110 can have a tapered lip 150 to guide the locking element 106 into the opening 108 or 110 during installation of the locking element 106 into the first and second components 102 and 104.

Referring again to FIG. 3, in an embodiment, the apparatus 100 can include an operable zone 152 in which the locking element 106 is adjustable within. In a particular instance, the locking element 106 can be moved between the locked and unlocked conditions within the operable zone 152. In an embodiment, the operable zone 152 is disposed on the outer surface 112 of the first component 102. In another embodiment, the operable zone 152 is disposed along the outer surface 114 of the second component 104. In a further embodiment, the operable zone 152 is disposed at a location other than the outer surfaces 112 and 114. In a particular embodiment, the operable zone 152 is disposed at a central location of the apparatus 100. In a more particular embodiment, the operable zone 152 is disposed at a central location of the outer surface 112 of the first component 102.

The operable zone 152 can include a feature, such as a guard 154. The guard 154 can define an outer periphery of the operable zone 152. In an embodiment, the guard 154 can extend from the outer surface 112 of the first component 102. The guard 154 can extend a distance from the outer surface 112 further than any portion of the locking element 106. In such a manner, the guard 154 can prevent the locking element 106 from contacting the skin of a patient. In an embodiment, the guard 154 can extend around the entire circumference of the operable zone 152. In another embodiment, the guard 154 can extend around less than the entire circumference of the operable zone 152. For example, the guard 154 can extend around at least 1% of the circumference of the operable zone 152 and no greater than 99% of the circumference of the operable zone 152. In an embodiment, the guard 154 can have a uniform shape or size as measured around the circumference of the operable zone 154. In another embodiment, the guard 154 can have a non-uniform shape or size as measured around the circumference of the operable zone 154.

In a non-illustrated embodiment, the operable zone 152 can be recessed from the outer surface 112 of the first component 102. In a particular instance, the operable zone 152 can be recessed by a dimension corresponding to a height of the exposed portion of the locking element 106. In such a manner, the locking element 106 can be recessed so as to not contact the skin of a patient during use or such that the locking element 106 is flush with the outer surface 112.

In an embodiment, one or more stops 156 can be disposed in the operable zone 152 to prevent over-rotation of the locking element 106. The one or more stops 156 can further prevent rotation of the locking element 106 in reverse rotational orientation (e.g., clockwise or counter-clockwise). In an embodiment, the one or more stops 156 can include at least one stop, or at least two stops, or at least three stops, or at least four stops, or even at least five stops. In embodiments with at least two stops, the stops can be equally spaced apart from one another around a circumference of the operable zone.

One or more stays 158 can be positioned on the apparatus 100. The one or more stays 158 can extend from the first component 102, the second component 104, or a combination thereof. In an embodiment, the one or more stays 158 can include two stays. The two or more stays 158 can have same or different sizes as compared to one another. In a particular embodiment, where the receiving portions 128 and 130 are different in size than the second receiving portions 132 and 134, the stays 158 can similarly be different in size to accommodate the different sized catheters. Insertion of the catheter into the stays 158 can enhance engagement thereof relative to the apparatus 100. Further, the stays 158 may redirect the catheters to a more suitable direction upon exiting the receiving portions 128, 130, 132, and 134. While not necessary for securement of the catheter to the apparatus 100, the stays 158 may act as a fail-safe to further secure the catheter and prevent undesirable movement thereof relative to the apparatus 100.

One or more engagement elements 160 can be disposed on the apparatus 100. The one or more engagement elements 160 can engage the apparatus 100 to a body. The one or more engagement elements 160 can optionally include openings through which line, such as suture, can be threaded to permit attachment of the apparatus 100 to the body. The line can be secured to the apparatus 100 prior to use with a neonate (e.g., during manufacture) or during engagement with the neonate (e.g., by a medical practitioner). By way of example, the line can be secured to an umbilical stump. In an embodiment, the apparatus 100 is adapted to be secured close to the umbilical stump (e.g., with a short length of line). The one or more engagement elements 160 can be positioned along a same side of the first or second component 102 or 104 to reduce line length when secured to an umbilical stump.

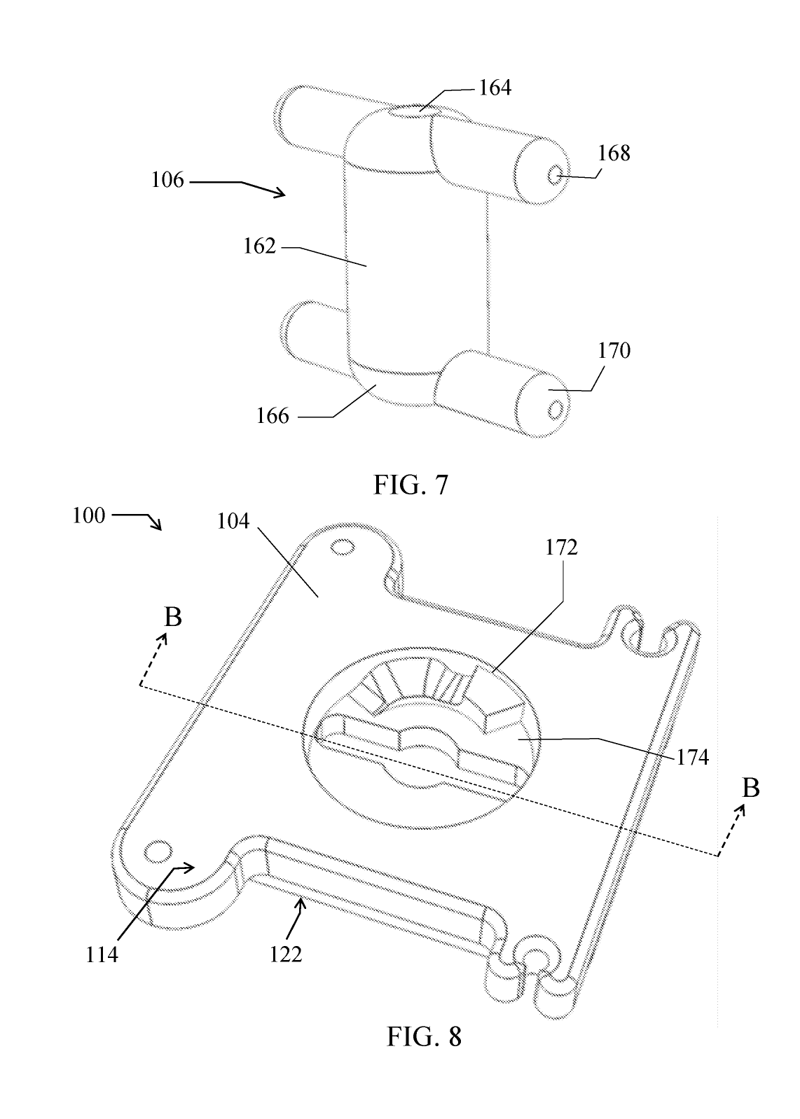

Referring now to FIG. 7, in an embodiment the locking element 106 includes a central segment 162 having a first longitudinal end 164 and a second longitudinal end 166. A first end segment 168 can be disposed adjacent to the first longitudinal end 164 of the central segment 162 and a second end segment 170 can be disposed adjacent to the second longitudinal end 166 of the central segment 166. In a particular embodiment, the first end segment 168 can be disposed at the first longitudinal end 164. In another particular embodiment, the second end segment 170 can be disposed at the second longitudinal end 166. The locking element 106 can include indicia or other markings along any one or more of the central segment 162, the first longitudinal end 164, and the second longitudinal end 166. The indicia or markings, for example, may facilitate easier alignment, engagement, or visual inspection of the apparatus 100.

In an embodiment, the first and second end segments 168 and 170 can lie along generally parallel lines. In another embodiment, the first and second end segments 168 and 170 can lie along generally parallel planes. At least one of the first and second end segments 168 and 170 can be oriented in a direction normal to the central segment 162. In an embodiment, at least one, such as all, of the central segment 162, first end segment 168, and second end segment 170 can include an elongated structure. In another embodiment, at least one, such as all, of the central segment 162, first end segment 168, and second end segment 170 can include a tubular structure. While the first and second end segments 168 and 170 are illustrated as generally similar structures in FIG. 7 (e.g., extending generally same directions and having a generally same shape and size), in an embodiment, the first and second end segments 168 and 170 can have different shapes, sizes, or orientations with respect to one another. For example, by way of a non-limiting embodiment, the first and second end segments 168 and 170 can be rotationally offset from one another by an angle in a range of 1° and 179°, or in a range of 5° and 160°, or in a range of 25° and 145°, or in a range of 60° and 120°, or in a range of 75° and 100°.

In an embodiment, the locking element 106 includes a central segment 162 and two end segments 168 and 170 having a maximum dimension, as measured normal to a length of the central segment 162, greater than a diameter, or largest dimension normal to the length of the central segment 162, of the central segment 162.

In an embodiment, the locking element 106 can be monolithic. In another embodiment, the locking element 106 can include a multi-component structure. In an embodiment, the locking element 106 can include one or more filler materials such as fibers. By way of non-limiting example, the filler materials can include carbon fibers or glass fibers. While a non-reinforced locking element 106 can have sufficient structural integrity to provide desired locking characteristic for the apparatus 100, in some embodiments a filler material may be suitable to enhance structural integrity of the locking element 106 and provide enhanced locking characteristic. In an embodiment, the locking element 106 can comprise a different material than at least one of the first and second components 102 or 104. In a more particular embodiment, the locking element 106 can include a material with higher strength as compared to the first or second components 102 or 104.

FIG. 8 illustrates a bottom perspective view of the second component 104. As illustrated, the second component 104 includes the outer surface 114 and inner surface 122 spaced apart from one another by a thickness of the second component 104. In an embodiment, the thickness can be at least 0.1 mm, or at least 0.2 mm, or at least 0.3 mm, or at least 0.5 mm, or at least 1 mm. In another embodiment, the thickness can be no greater than 10 cm, or no greater than 5 cm, or no greater than 1 cm, or no greater than 5 mm. In an embodiment, the outer surface 114 and inner surface 122 can be generally parallel with respect to one another. In an embodiment, the outer surface 114 includes an operable zone 172 adapted to receive the locking element 106 in combination with the operable zone 152 of the first component 102 to secure the apparatus 100 and a catheter therein.

In an embodiment, the operable zone 172 includes a recessed portion 174 extending into the second component 104 from the outer surface 114. In another embodiment, the operable zone 172 includes a guard similar to the guard 154 described above. In yet a further embodiment, the operable zone 172 includes a guard and a recessed portion.

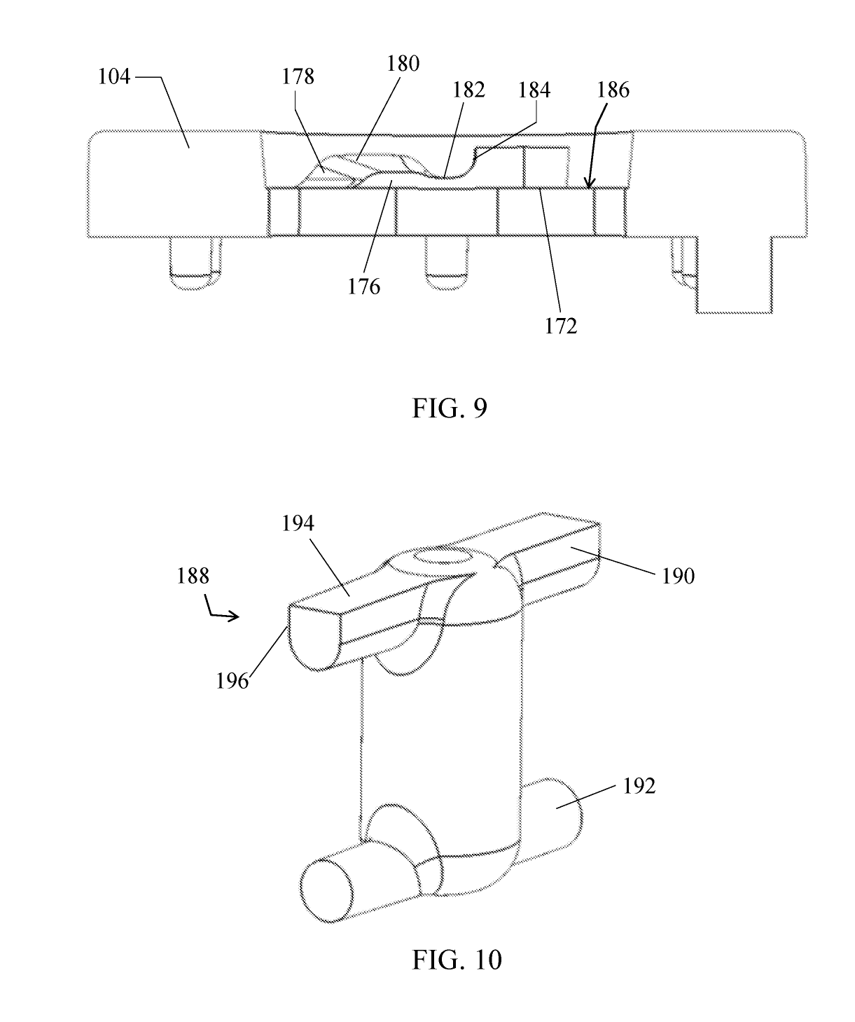

Referring to FIG. 9, the operable zone 172 can include a locking component 176 adapted to receive a portion of the locking element 106 (FIG. 7). The locking component 176 can secure the locking element 106 at a fixed position relative to the second component 104. In an embodiment, the locking component 176 includes a ramped portion 178 defining an apex 180, a recessed portion 182, and a stop 184. In an embodiment, the end of the ramped portion 178 is adjacent to the apex 180. In another embodiment, the apex 180 is adjacent to the recessed portion 182. In a further embodiment, the recessed portion 182 is adjacent to the stop 184. The stop 184 can prevent overrotation of the locking element 106 in embodiments where the locking element 106 translates between a locked and unlocked position via rotational movement.

The locking element 106 can rotate such that one of the first and second end segments 168 or 170 moves along the ramped portion 178 to the apex 180 and into the recessed portion 182. The first or second end segment 168 or 170 can rotate within the recessed portion 182 until contacting the stop 184. In a particular embodiment, the locking element 106, the locking component 176, or a combination thereof can create a tactile or audible indication to a user when the locking element 106 is secure within the recessed portion 182 and the apparatus 100 is fully closed. For example, the locking element 106, the locking component 176, or a combination thereof can generate an audible sound, a physical feedback, or a combination thereof when the apparatus 100 is fully closed (i.e., when the first or second end segment 168 or 170 is positioned in, or substantially in, the recessed portion 182).

In an embodiment, the locking component 176 includes a variable height, as measured from a bottom surface 186 of the locking component 176, such as a first height at the apex 180, a second height at the recessed portion 182, and a third height at the stop 184. In a particular embodiment, the first height is greater than the second height. In another embodiment, the third height is greater than the second height. In a further embodiment, the third height is greater than the first height.

The ramped portion 178 can be disposed at a relative angle, as measured with respect to the bottom surface 186, of at least 1°, or at least 2°, or at least 3°, or at least 4°, or at least 5°, or at least 6°, or at least 7°, or at least 8°, or at least 9°, or at least 10°. In an embodiment, the ramped portion 178 can have a relative angle, as measured with respect to the bottom surface 186, of no greater than 60°, or no greater than 30°, or no greater than 20°, or no greater than 15°. In a particular embodiment, the apex 180 is rounded. In another particular embodiment, the apex comprises a junction to two or more linear sections joined at relative angles.

FIG. 10 illustrates another embodiment of a locking element 188. The locking element 188 can include a first end segment 190 and a second end segment 192. The first segment 190 can have a non-rounded cross-section and the second segment 192 can have a rounded cross-section. As illustrated, the first end segment 190 can include a generally planar upper surface 194. In another embodiment, the first end segment 190 can include one or more planar, or generally planar or partially planar, side surfaces 196.

In certain embodiments, the locking element is moved between an unlocked and locked configuration by manual operation, for example, using a vice such as a hemostat or other similar device. Without wishing to be exclusively bound by any particular theory, it is believed that a non-rounded first end segment 190 may enhance engagement with the vice, permitting easier grip and operation therewith. In other embodiments, the first end segment 190 can include tabs, ridges, castellations, surface roughness, thinned portions, projections or tines, engagement mechanisms adapted to be grabbed by specialized tooling, or any combination thereof to permit easier manual operation during locking and unlocking.

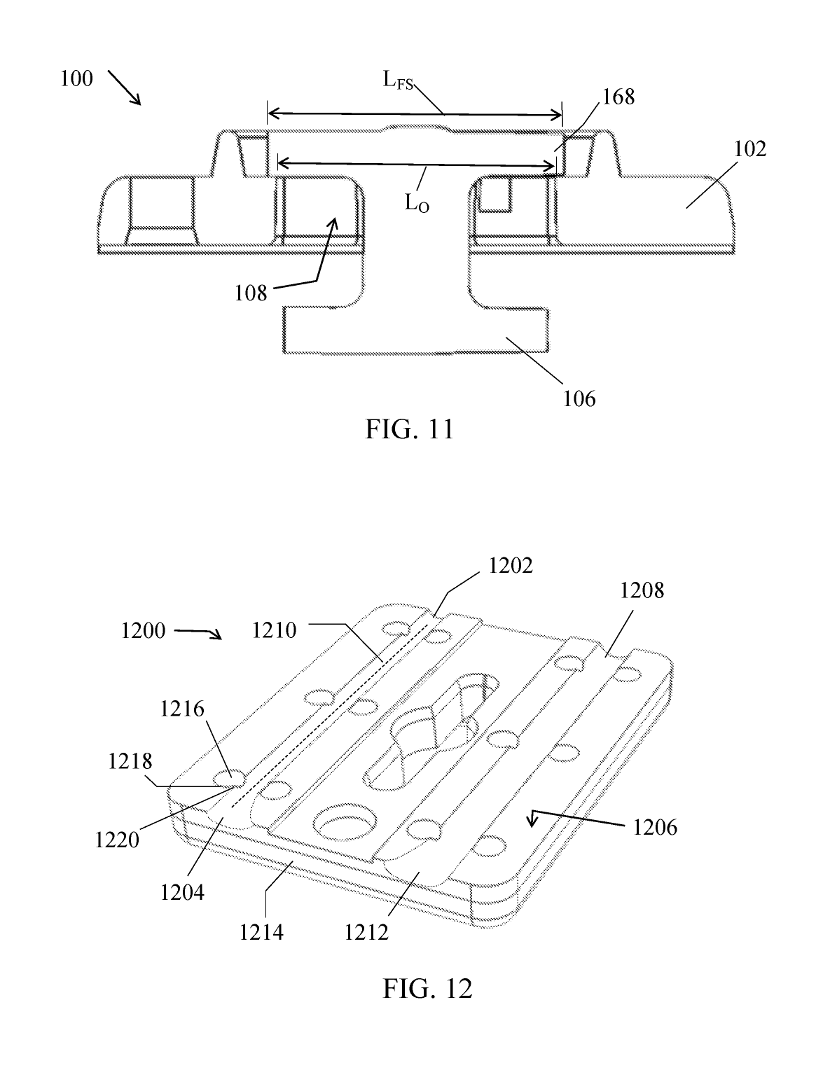

FIG. 11 illustrates an exemplary cross-sectional view of the apparatus 100, including the first component 102 and the locking element 106. As illustrated, the opening 108 has a length, L_{O}, that is less than the length, L_{FS}, of the first segment 168 of the locking element 106. In an embodiment, L_{O} is no greater than 0.99 L_{FS}, or no greater than 0.98 L_{FS}, or no greater than 0.97 L_{FS}, or no greater than 0.96 L_{FS}, or no greater than 0.95 L_{FS}, or no greater than 0.9 L_{FS}, or no greater than 0.8 L_{FS}, or no greater than 0.7 L_{FS}, or no greater than 0.6 L_{FS}, or no greater than 0.5 L_{FS}, or no greater than 0.4 L_{FS}. In another embodiment, L_{O} is no less than 0.01 L_{FS}, or no less than 0.2 L_{FS}, or no less than 0.3 L_{FS}.

In another embodiment, the length, L_{O}, of the opening 108 is the same, or approximately the same, as the length, L_{FS}, of the first segment 168. In yet a further embodiment, the length, L_{O}, of the opening 108 is greater than the length, L_{FS}, of the first segment 168.

FIG. 12 includes another embodiment of the first component 1200 including a receiving portion 1202 having a flared end 1204. In an embodiment, the flared end 1204 can have a linear taper. That is, the diameter of the flared end can change along a linear path. In another embodiment, the flared end 1204 can have an arcuate taper. In yet a further embodiment, the flared end 1204 can have a taper including one or more linear portions and one or more arcuate portions.

A non-flared portion of the receiving portion 1202 can have a width, W_{RP}, as measured, for example, along a plane defined by an inner surface 1206 of the first component, that is less than a width, W_{FE}, of the flared end 1204, as measured, for example, along a plane defined by an inner surface 1206 of the first component. In an embodiment, W_{FE} is at least 1.01 W_{RP}, or at least 1.02 W_{RP}, or at least 1.03 W_{RP}, or at least 1.04 W_{RP}, or at least 1.05 W_{RP}, or at least 1.1 W_{RP}, or at least 1.2 W_{RP}, or at least 1.3 W_{RP}, or at least 1.4 W_{RP}, or at least 1.5 W_{RP}. In another embodiment, W_{FE} is no greater than 10.0 W_{RP}, or no greater than 8.0 W_{RP}, or no greater than 5.0 W_{RP}, or no greater than 2.0 W_{RP}.

In the illustrated embodiment, the flared end 1204 flares inward, toward a second receiving portion 1208 of the first component 1200. In another embodiment, the flared end 1204 can flare outward, away from the second receiving portion 1208. In yet a further embodiment, the flared end 1204 can flare inward and outward. In a more particular embodiment, the inward portion of the flared end 1204 can extend further from a central axis 1210 of the receiving portion 1208 than the outward portion of the flared end 1204 extends from the central axis 1210. In another more particular embodiment, the outer portion of the flared end 1204 can extend further from the central axis 1210 of the receiving portion 1208 than the inward portion of the flared end 1204. In yet a further particular embodiment, the inner and outer portions of the flared end 1204 can be symmetrical about the central axis 1210.

The flared end 1204 can extend along at least 0.5% the length of the receiving portion 1204, or along at least 1% of the length of the receiving portion 1204, or along at least 2% of the length of the receiving portion 1204, or along at least 3% of the length of the receiving portion 1204, or along at least 5% of the length of the receiving portion 1204, or along at least 10% of the length of the receiving portion 1204. In an embodiment, the flared end 1204 can extend along no greater than 75% of the length of the receiving portion 1204, or no greater than 50% of the length of the receiving portion 1204, or no greater than 25% of the length of the receiving portion 1204, or no greater than 15% of the length of the receiving portion 1204.

In an embodiment, the flared end 1204 can extend from an outer edge 1214 of the first component 1200 toward a nearest recess (or post) 1216. In a more particular embodiment, the flared end 1204 can extend from the outer edge 1214 to an outer edge 1218 of the nearest recess (or post) 1216. In yet a more particular embodiment, the flared end 1204 can terminate at the outer edge 1218 of the nearest recess (or post) 1216. In another embodiment, the flared edge 1204 can terminate prior to the outer edge 1218 of the nearest recess (or post) 1216. In yet another embodiment, the flared edge 1204 can extend from the outer edge 1214 past the outer edge of the nearest recess (or post) 1216. The flared edge 1204 can extend to a contact point 1220 with the nearest recess (or post) 1216. In an embodiment, the contact point 1220 can be angularly offset from the outer edge 1214 of the nearest recess (or post) 1216 by at least 1°, or at least 2°, or at least 3°, or at least 4°, or at least 5°, or at least 10°, or at least 15°, or at least 20°, or at least 25°, or at least 30°, or at least 35°, or at least 40°, or at least 45°. In another embodiment, the contact point 1220 can be angularly offset form the outer edge 1214 by no greater than 120°, no greater than 90°, or no greater than 75°.

In an embodiment, the second receiving portion 1208 can included a flared end 1212. The flared end 1212 can have similar or different attributes as compared to the flared end 1204. In an embodiment, the flared ends 1204 and 1212 can flare, or extend, towards one another. In a more particular embodiment, the flared ends 1204 and 1212 can be contiguous with one another. The flared ends 1204 and 1212 can form a continuous flared portion extending between the receiving portions 1202 and 1208.

In a particular instance, the flared ends 1204 and 1212 can permit flexure of catheters at the ends of the receiving portions 1202 and 1208. Flexure of the catheters can prevent kinking or binding which might otherwise occur in embodiments where the receiving portions 1202 and 1208 have terminating widths equal to the width of the catheter. In operation, because of the close proximity of umbilical catheters within the umbilical stump, use of two catheters generally requires inward catheter flexure. Flared ends 1204 and 1212 can prevent binding that can occur because of such flexure.

In an embodiment, the thickness of at least one of the receiving portions 1202 or 1208 can be uniform along the entire length of the receiving portion 1202 or 1208. That is, the flared end 1204 or 1212 can flare only in one plane. More particularly, the flared end 1204 or 1212 can flare in a plane generally coplanar with a plane formed by the inner surface 1206. In another embodiment, the thickness of at least one of the receiving portions 1202 or 1208 can be different at a central location of the receiving portion 1202 or 1208 and the flared end 1204 or 1212. For example, the flared end 1202 or 1208 can flare in multiple planes (i.e., perpendicular planes). In a particular embodiment the flared end 1202 or 1208 can have a uniform, or generally uniform, flare as measured around the circumference, or outer edge, of the flared end 1202 or 1208.

In certain embodiments, both longitudinal ends of at least one of the receiving portion 1204 and second receiving portion 1208 can include flared ends. Both flared ends on the same receiving portion can have a same shape or a same size as compared to one another. In a particular instance, the flared ends on opposite longitudinal ends of the receiving portion can have different shapes or different sizes.

In other embodiments, only one of the longitudinal ends of at least one of the receiving portion 1204 and second receiving portion 1208 can include a flared end.

In an embodiment, an apparatus 100 described in accordance with an embodiment herein can receive a catheter. The catheter can be secured to the apparatus 100. In an embodiment, the catheter can be secured to the apparatus 100 such that the catheter does not move relative to the apparatus 100. For example, the apparatus 100 can prevent longitudinal translation of the catheter, rotational movement of the catheter, or both longitudinal translation and rotational movement of the catheter relative to the apparatus. In an embodiment, the apparatus is adapted to prevent relative movement of the catheter when a longitudinal biasing force is applied to the catheter. For example, the apparatus 100 can be adapted to prevent longitudinal translation of the catheter upon application of a longitudinal force of at least 1 N, or at least 2 N, or at least 3 N, or at least 5 N, or at least 10 N, or at least 50 N, or at least 100 N. In another embodiment, the apparatus 100 is adapted to prevent longitudinal translation of the catheter upon application of no greater than 10,000 N of force. Projections such as fingers, tines, dimples, waves, or any combination thereof positioned within the receiving portions can further prevent relative movement of the catheter with respect to the apparatus 100.

In an embodiment, the apparatus is adapted to apply a relatively even force to the catheter, as measured around a circumference thereof, during operation. As used herein, “a relatively even force” refers to a condition whereby the force provided by the apparatus to the catheter along the circumference of the catheter at any single location differs from an average force, as measured by an average force at all, or substantially all, locations around the catheter, by no greater than 10 N/mm², or no greater than 5 N/mm², or no greater than 1 N/mm². In a particular embodiment, the apparatus is adapted to apply even force to the catheter, as measured around the circumference thereof. As used herein, “even force” refers to a condition whereby the force provided by the apparatus to the catheter along the circumference of the catheter at any single location differs from the average force, as measured by an average force at all locations around the catheter, by no greater than 0.1 N/mm², or no greater than 0.01 N/mm², or no greater than 0.001 N/mm².

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Separate embodiments may also be provided in combination in a single embodiment, and conversely, various features that are, for brevity, described in the context of a single embodiment, may also be provided separately or in any subcombination.

Further, reference to values stated in ranges includes each and every value within that range. Many other embodiments may be apparent to skilled artisans only after reading this specification. Other embodiments may be used and derived from the disclosure, such that a structural substitution, logical substitution, or another change may be made without departing from the scope of the disclosure. Accordingly, the disclosure is to be regarded as illustrative rather than restrictive.

1. An apparatus comprising:
a first component;
a second component; and
a locking element adapted to selectively couple the first and second components together, wherein the locking element is moveable between a first position and a second position, wherein the apparatus is adapted to secure a catheter between the first and second components when the locking element is in the first position, and wherein the catheter is moveable with respect to the apparatus when the locking element is in the second position.

2. The apparatus of claim 1, wherein the locking element is adapted to translate relative to at least one of the first and second components in a direction normal to a major surface of the first component during rotation between the first and second positions.

3. The apparatus of claim 1, wherein the first and second components define a receiving portion adapted to receive and secure the catheter, and wherein the receiving portion has a flared end.

4. The apparatus of claim 3, wherein the first component comprises a plurality of posts extending from an inner surface, and wherein an innermost surface of at least one of the plurality of posts is contiguous with the receiving portion.

5. The apparatus of claim 4, wherein the plurality of posts is adapted to maintain the catheter in close proximity to the receiving portion when the locking element is in the second position.

6. The apparatus of claim 1, wherein the first and second components are spaced apart from one another when the locking element is in the first position.

7. The apparatus of claim 1, wherein the locking element comprises:
a central segment;
a first end segment disposed at a first longitudinal end of the central segment; and
a second end segment disposed at a second longitudinal end of the central segment,
wherein the first and second segments are adapted to contact outer surfaces of the first and second components, respectively, when the locking element is in the first position.

8. The apparatus of claim 1, wherein the locking element comprises a first material, wherein the first and second components comprise a second material, and wherein the first material is more resistant to deformation than the second material.

9. The apparatus of claim 1, wherein the locking element is not readily removable from an opening in the first component.

10. An apparatus comprising:
a first component;
a second component; and
a locking element adapted to selectively couple the first and second components together,
wherein the apparatus is adapted to selectively secure a catheter between the first and second components, and wherein nearest major surfaces of the first and second components are spaced apart from one another when the catheter is selectively secured to the apparatus.

11. The apparatus of claim 10, wherein the catheter is visible through a gap between the first and second components when the catheter is secured to the apparatus.

12. The apparatus of claim 10, wherein the locking element is adapted to selectively move between a first position and a second position, wherein the apparatus is adapted to secure a catheter at a relatively fixed position with respect to the apparatus when the locking element is in the first position, and wherein the catheter is moveable with respect to the apparatus when the locking element is in the second position.

13. The apparatus of claim 10, wherein the apparatus is adapted to provide a tactile indication to a user when the locking element is locked.

14. The apparatus of claim 10, wherein the apparatus comprises a receiving portion adapted to receive and secure the catheter between the first and second components, and wherein the receiving portion has a flared end.

15. The apparatus of claim 14, wherein the apparatus further comprises a second receiving portion adapted to receive and secure a second catheter between the first and second components, wherein the second receiving portion has a flared end, and wherein the flared ends of the first and second receiving portions are contiguous with one another.

16. The apparatus of claim 10, wherein the locking element is centrally disposed along the first component and extends through at least one of the first and second components.

17. The apparatus of claim 10, wherein the first component comprises a first alignment element, wherein the second component comprises a second alignment element, and wherein the first and second alignment elements are complimentary, and wherein the first and second alignment elements are adapted to prevent improper assembly of the first and second components with respect to one another.

18. An apparatus adapted to secure a catheter to a body, wherein the apparatus is adapted to provide substantially equal compressive force to the catheter along a substantial entire engagement interface of the apparatus and catheter, wherein the apparatus is selectively lockable by a locking element, and wherein the apparatus is adapted to secure the catheter to the body without adhesive material.

19. The apparatus of claim 18, wherein:
the apparatus comprises a receiving portion adapted to receive and secure the catheter,
the receiving portion includes a first receiving portion on a first component of the apparatus and a second receiving portion on a second component of the apparatus,
the first component comprises a plurality of posts disposed immediately adjacent to the first receiving portion,
the second component comprises a plurality of recesses disposed immediately adjacent to the second receiving portion, and
the plurality of posts is adapted to be received in the plurality of recesses when the apparatus is in a locked position.

20. The apparatus of claim 19, wherein the plurality of posts is adapted to maintain the catheter in close proximity to the first receiving portion when the apparatus is in an unlocked position.
